(19) United States
(12) Patent Application Publication
(10) Pub. No.: US 2004/0028572 A1
(43) Pub. Date: Feb. 12, 2004

(54) Ozone deodorizer for waste receptacles

(76) Inventors: John C.K. Sham, Hong Kong (HK); Kumkit Kunavongvorakul, Sathorn Bangkok (TH)

Correspondence Address: Peter S. Canelias, Attorney at Law, 14th Floor, 445 Park Avenue, New York, NY, 10022, US

(21) Appl. No.: 10/217,063

(22) Filed: Aug. 12, 2002

(51) Int. Cl. A61L 9/015

(52) U.S. Cl. 422/123; 422/305; 422/306

(57) ABSTRACT

A deodorizing/odor removal and disinfection device designed to function in conjunction with a waste receptacle such as a garbage can. The device is equipped with an ozone/ion generator that generates an ozone/ion laden air stream and introduces the air stream into the interior of the waste receptacle without unduly interfering with the operation or seating of the lid of the receptacle. A securing mechanism such as a hook secures the deodorizing device to the exterior of the waste receptacle by engaging with the upper edge or lip of the receptacle, and preferably uses a tube to transmit the ozone laden air stream into the malodorous environment.

### BACKGROUND OF THE INVENTION

The invention relates to a deodorizing and disinfecting device and method, and more particularly, to a portable, preferably detachable deodorizing and disinfecting device for deodorizing and disinfecting a waste receptacle such as a garbage can. Particles, odors, bacteria, microorganisms and other contaminants are frequently unpleasant and sometimes unhealthy to breathe. Vapors are formed from liquids or solids, and living organisms, such as bacteria, are also contaminants. Household garbage typically contains one or more of these malodorous components. When accompanied by bacterial action these wastes make putrefaction and decay a continual odor source and nuisance in the indoor environment.

Household odors, such as those stemming from garbage containers, are difficult to control because they are many and different in source, and the odors typically disperse from the source throughout the ambient atmosphere. Various methods have been employed to control malodors, including various filtration devices, aerosols, solid air fresheners, liquids, powders, granules and gases. These attempts may be divided into purification and odor masking methods.

Odor masking superimposes one malodor on another stronger odor, thereby creating a new and often unpleasant odor. The method is typically used in toilet and waste disposal areas, and many users do not like the odors produced from the materials which are usually chemically derived. And, this method only lasts as long as a continuous supply of the masking odor is supplied, and requires placing chemicals, fogs, mists or sprays into the ambient air.

Chemical reactions require deodorizers that provide a lasting effect, and they are usually chemical concentrates in liquid, block and granular forms. These methods are used mostly because they are low in cost, but usually are pollutants themselves because of the chemical fumes and gases that are released. Biological methods of combating odor, such as using enzymes and bacteria to break down odor causing matter, are not always effective. Enzymes, for example, are limited in their use and versatility, work on a limited variety of malodorous substances, and are slow acting.

Filters and electronic air cleaners are often used to clean air in large areas. These devices include air exchange filtration systems, or electronically charged fields and plates to collect and remove particle matter. Although often effective for larger areas, they are costly to use and maintain. And, they fail to target odors at the source, relying instead on a broader air purification of the ambient atmosphere.

A known deodorant and disinfectant is ozone, also referred to as triatomic oxygen. “Ozonation” is one of many methods used for the purification of diverse substances, especially water. It is a technology substantially more effective than odor masking methods, because ozone not only can kill bacteria and viruses, but also can oxidize organics such as garbage waste, and inorganics such as iron, manganese, organically bound heavy metals, cyanides, sulfides, and nitrates. Ozone is an unstable gas having life in water of minutes. Oxygen, which is normally bi-atomic, becomes ozone through the addition of a third unstable atom, giving the compound a life in water of only minutes. Ozone is created by either of two generation methods: ultraviolet radiation or corona discharge. Of the two, corona discharge produces the substantially higher ozone concentration needed for the removal of complex impurities.

Devices that produce ozone via electrolysis and employ ozone for deodorization and/or disinfecting are known in the art. For example, U.S. Pat. No. 6,134,806 describes an apparatus for sterilizing and deodorizing a portable sports equipment bag utilizing filters and an ozone generator and blower. Many of these devices, however, are not suited to particular applications, are of complex construction and comprise parts which are not readily available. Many electrolytic devices for producing ozone are large, expensive and difficult to transport, as well as complicated enough to preclude operation by unskilled individuals. And, it is not desirable that ozone be generated over a wide area, but rather directed specifically to the object to be deodorized or disinfected.

In all of the prior methods and devices for deodorization, however, there are drawbacks. The prior methods are either inefficient, i.e., mask rather than remove odors, or are suited to cleaning the atmosphere of a large area, such as a room. The prior methods devices are ill-adapted to attacking odors-at the most likely source in order to prevent them from dispersing into the ambient atmosphere. None of them are particularly adapted to a waste receptacle, such as a garbage can. The present invention solves these and other problems as will be made apparent.

### BRIEF SUMMARY OF THE INVENTION

The present invention is a compact, self contained ozone generator and deodorization unit that may be fitted onto a waste receptacle without unduly interfering with the operation of the receptacle lid, if any. The design is such that by hanging from the upper edge or lip of a substantially rigid waste receptacle, a stream of ozone is transmitted via an air tube to infuse the air within the waste receptacle, i.e., the air flow is enriched with ozone or “ozonated.” This function allows the localization of the ozone laden air within the receptacle, rather than wide-area dispersion of ozone into the ambient atmosphere. The invention also provides easy means of attaching and removing the device, such as a hanger which fits over the upper edge of the waste receptacle. The use a hanger or hook, combined with a flexible, deformable transmission tube, allow for the device to be used in garbage cans without interfering with the normal operation of the lid, whether the lid is movable by hand or by means of a foot pedal.

It is therefore an object of this invention to provide a deodorizing mechanism that removes odors from a waste receptacle.

It is a further object of this invention to provide a deodorizing mechanism that employs ozone or ion generation to remove odors from a waste receptacle.

It is a further object of this invention to provide a deodorizing mechanism that is easily attached and detached from a waste receptacle.

It is a further object of this invention to provide a deodorizing mechanism that delivers a stream of ozone or ion laden air to the interior of a waste receptacle.

It is a further object of this invention to provide a deodorizing mechanism that disinfects the interior of a waste receptacle by means of an ozone laden air stream.

It is a further object of this invention to provide a deodorizing mechanism that delivers a stream of ozone or ion laden air to the interior of a waste receptacle without unduly interfering with the operation of a lid covering the waste receptacle.

It is a further object of this invention to provide a deodorizing mechanism that delivers a stream of ozone or ion laden air to the interior of a waste receptacle rather than wide-area dispersion of ozone into the ambient atmosphere.

It is a further object of this invention to provide a deodorizing mechanism that removes odors from a waste receptacle without requiring the changing of filters or filter media.

It is a further object of the invention to provide a deodorizing mechanism that may be easily maintained.

It is a further object of the invention to direct the path of an incoming ozone or ion laden air stream so that substantially all of the particulate matter is precipitated within the interior of the receptacle and substantially remains entrapped within the receptacle.

Other objects and advantages will be more fully apparent from the following disclosure and appended claims.

### BRIEF DESCRIPTION OF THE DRAWINGS

The above-mentioned and other features and objects of this invention and the manner of obtaining them will become apparent and the invention itself will be best understood by reference to the following description of an embodiment of the invention taken in conjunction with the accompanying drawings, wherein:

FIG. 1 is a perspective view of an embodiment of the invention engaged on a waste receptacle;

FIG. 2 is a view of an embodiment of the deodorization unit;

FIG. 3 is a view of a support frame for supporting the deodorization unit;

FIG. 4 is a view of the deodorization unit engaged with the support frame;

FIG. 5 is a view of a receptacle with stabilizer feet to compensate for the deodorization unit;

FIG. 6 is a perspective view of an alternative embodiment of the deodorization unit;

FIG. 7 is a rear perspective view of an alternative embodiment of the deodorization unit;

FIG. 8 is a front view of an alternative embodiment of the deodorization unit;

FIG. 9 is a side sectional view of a receptacle showing the air flow from the deodorization unit into the interior of the receptacle;

FIG. 10 is a view of an ozone generator assembly;

FIG. 11 is an exploded view of the ozone generator assemble.

### DETAILED DESCRIPTION

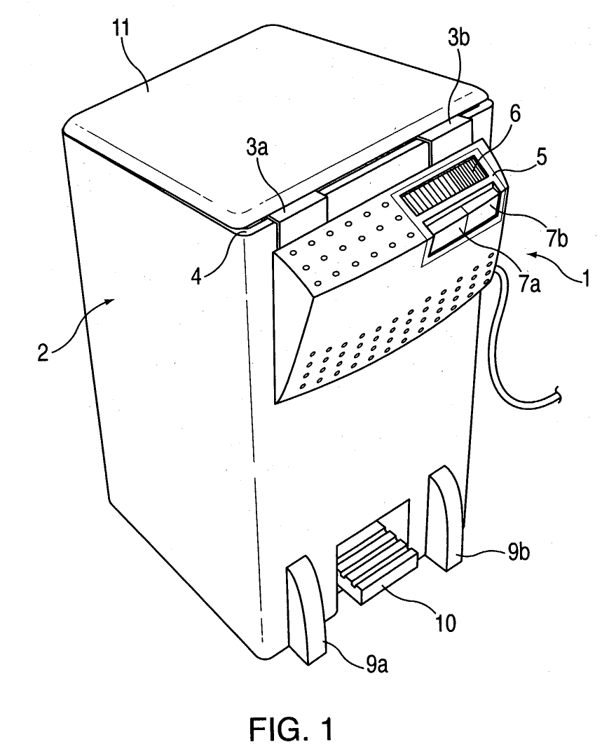

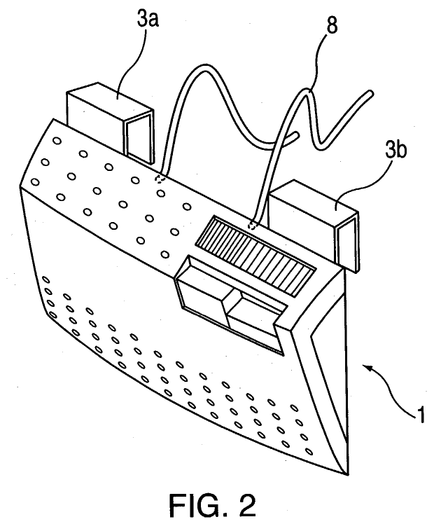

Turning now to the drawings, FIG. 1 shows the deodorization unit 1 detachably secured to a waste receptacle 2 by means of two hanger clips or hooks 3a, 3b engaged over the upper lip 4 of the receptacle 2. The deodorization unit 1 has a display panel 5 that may be employed to monitor the status of the unit such as an indicator light 6 to indicate whether the power supply to the unit is on or off. Buttons 7a, 7b may be provided to vary the speed of flow from the unit to two or more speeds. FIG. 2 shows the deodorization unit 1 with the flow tube 8 for introducing an ozone laden air stream into the interior of the receptacle 2. The tube 8 may also be carried by means of a hook or hooks 3a, 3b, to support the tube 8 as it extends into the interior of the receptacle 2. Preferably, the tube 8 should be sized so as not to unduly interfere with the seating or operation of a lid 11 that covers the receptacle 2. Preferably, the tube 8 should be formed of a flexible, deformable material such as rubber or plastic so that the flow of ozone laden air may continue unimpeded even where the tube 8 is compressed or impinged between the lid 11 and the upper edge 4 of the receptacle 2. Although a slight gap may be created between the lid 11 and the upper edge 4 of the receptacle 2, such gap will not substantially allow the escape of malodors or excess amounts of ozone from the receptacle 2.

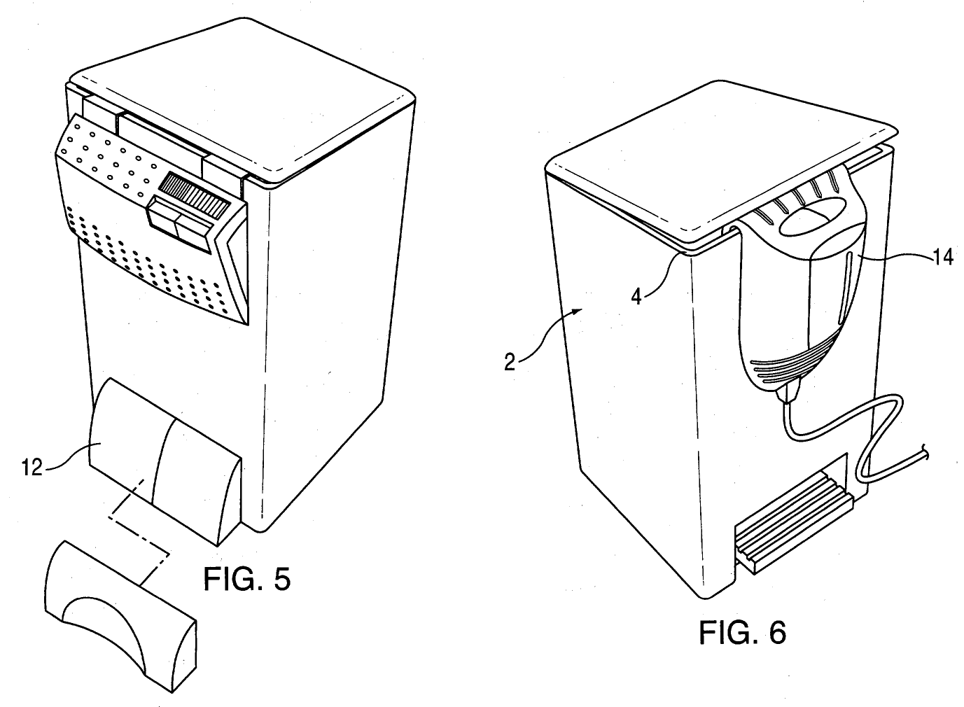

The deodorization unit 1 may require support in order to maintain the stability of the receptacle 2. FIG. 1 shows two stabilizer feet 9a and 9b, located on either side of depressible foot lever 10. Depressible foot lever 10 is used to raise and lower the lid 11 by depressing or removing pressure, respectively, on or from the foot lever 10. The stabilizer feet 9a and 9b should be situated so as not to interfere with the operation of the depressible foot lever 10, and separated from each other by a distance sufficient to prevent the receptacle 2 from toppling when the weight of the deodorization unit 1 is added to the upper portion of the receptacle 2. FIG. 5 shows a different configuration employing a wider single foot 12 where access to a depressible foot lever is unnecessary.

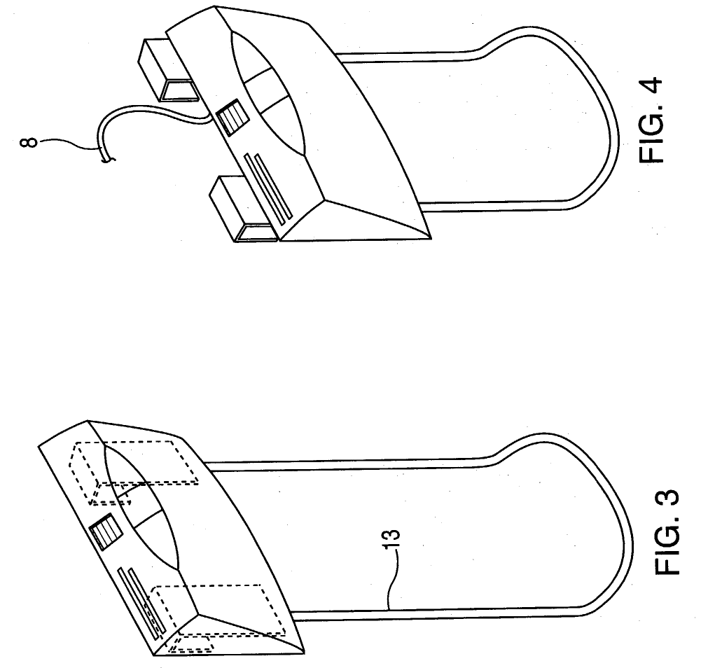

FIG. 3 shows a support frame 13 for engagement with the deodorization unit 1. When engaged with the deodorization unit 1 (see FIG. 4) the support frame 13 redistributes the weight of the deodorization unit 1 to points lower along the front side of receptacle 2, contributing to a lower center of gravity and reduction of the likelihood of toppling.

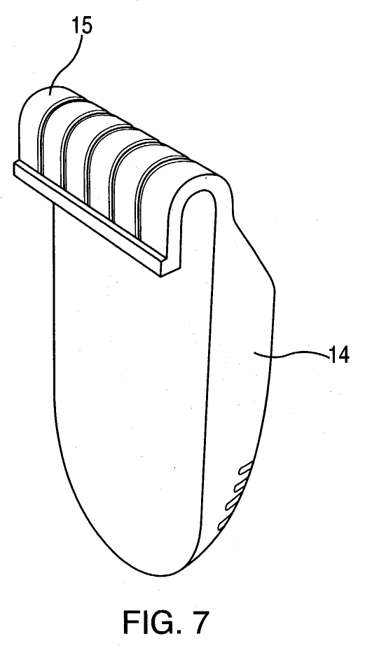

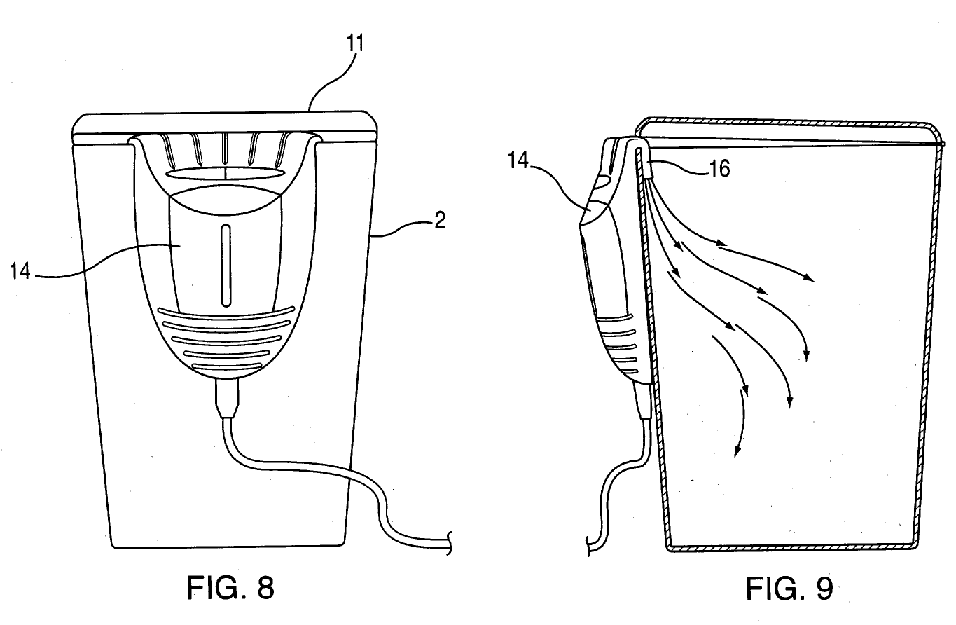

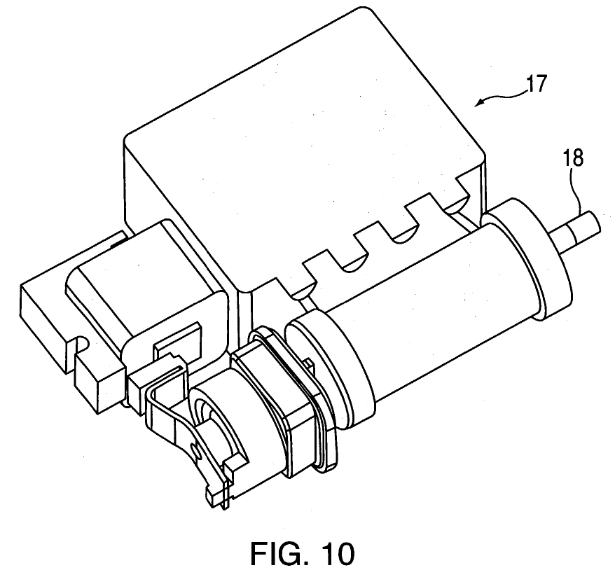

FIGS. 6-9 show an alternative embodiment of the invention, where the deodorization unit 14 is of a more compact design, and employs a single hanging clip or hook 15 (FIG. 7), here shown in the form of a claw hook, to engage with the top edge or lip 4 of the receptacle 2. FIG. 9 shows the passage of the ozone laden air flow stream from the deodorization unit 14 through tube 16 into the interior of the receptacle 2.

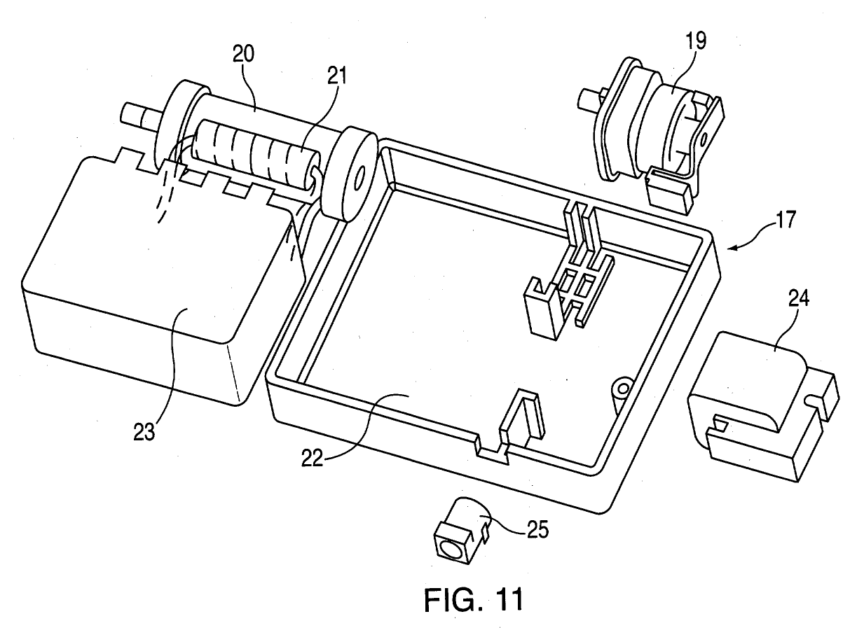

FIG. 10 shows a preferred ozone generator assembly 17 for use in the present invention. It is understood by those of skill in the art that a negatively-charged ion may be substituted for ozone. An ozone/ion outlet 18, is in fluid communication with the tube 8 (FIG. 2) for introducing an ozone or ion laden air stream into the interior of the receptacle 2. FIG. 11 shows the workings of the ozone generator assembly 17, having an air pump assembly 19 for introducing an air flow though the air duct 20 in communication with the ozone tube 21 for forming ozone laden air which is then discharged from outlet 18 and into the tube 8 (FIG. 2) for introducing an ozone or ion laden air stream into the interior of the receptacle 2.

The main housing 22 of the ozone generator assembly 17 is preferably formed of impact-resistant plastic, which is slotted and formed as appropriate to retain the various components comprising the ozone generator assembly 17. The power source for the ozone generator assembly 17 is preferably AC (alternating current), preferably standard household current, but a DC (direct current) power source may be employed where the availability of a suitable AC outlet or power supply is either uncertain or absent entirely. The high-voltage transformer 23 receives and steps up the voltage from an external source through power jack 25 in order to generate ozone. An electromagnet 24 is connected to the air pump assembly 19 to deliver an ozone laden air stream into the tube 8 (FIG. 2) and from the tube 8 into the interior of the receptacle 2.

Since other modifications or changes will be apparent to those skilled in the art, there have been described above the principles of this invention in connection with specific apparatus, it is to be clearly understood that this description is made only by way of example and not as a limitation to the scope of the invention.

What is claimed is:

1. An apparatus for deodorization comprising:
a main housing;
an ozone generator carried on said main housing;
a power source for said ozone generator;
a tube extending from said ozone generator for transmitting ozone to a receptacle to be deodorized;
a fastener for securing said main housing and said ozone generator to a receptacle to be deodorized.

2. The apparatus according to claim 1, wherein said fastener is a hook for releasably securing said main housing and said ozone generator to said receptacle to be deodorized.

3. The apparatus according to claim 1, wherein said main housing is engaged with a supporting frame for stabilizing said main housing on an exterior surface of said receptacle.

4. The apparatus according to claim 1, wherein said tube is carried on said fastener.

5. The apparatus according to claim 1, wherein said tube is formed of a flexible material.

6. An apparatus for deodorization comprising:
a main housing;
an ozone generator carried on said main housing;
a power source for said ozone generator;
a receptacle having an upper edge;
a tube extending from said ozone generator to an interior of said receptacle;
a fastener for securing said main housing and said ozone generator to said upper edge of said receptacle.

7. The apparatus according to claim 6, wherein said main housing is engaged with a supporting frame for stabilizing said main housing on an exterior surface of said receptacle.

8. The apparatus according to claim 6, wherein said fastener is a hook for releasably securing said main housing and said ozone generator to said receptacle to be deodorized.

9. The apparatus according to claim 6, wherein said tube is carried on said fastener.

10. The apparatus according to claim 6, wherein said tube is formed of a flexible material.

11. The apparatus according to claim 6, wherein said receptacle carries a stabilizing foot at a lower portion of said receptacle to substantially stabilize said receptacle.

12. An apparatus for deodorization comprising:
a main housing;
an ozone generator carried on said main housing;
said ozone generator having an air pump for pumping air through said ozone generator;
a power source for said ozone generator;
a tube extending from said ozone generator for transmitting ozone to a receptacle to be deodorized;
a fastener for securing said main housing and said ozone generator to a receptacle to be deodorized.

13. The apparatus according to claim 12, wherein said fastener is a hook for releasably securing said main housing and said ozone generator to said receptacle to be deodorized.

14. The apparatus according to claim 12, wherein said main housing is engaged with a supporting frame for stabilizing said main housing on an exterior surface of said receptacle.

15. The apparatus according to claim 12, wherein said tube is carried on said fastener.

16. The apparatus according to claim 12, wherein said tube is formed of a flexible material.
